(19) United States
(12) Patent Application Publication
(10) Pub. No.: US 2026/0007781 A1
(43) Pub. Date: Jan. 8, 2026

(54) PROSTATE SPECIFIC MEMBRANE ANTIGEN (PSMA) LIGANDS AND USES THEREOF

(71) Applicant: Novartis AG, Basel (CH)

(72) Inventors: Donato Barbato, Ivrea (IT); Maurizio F. Mariani, Ivrea (IT); Francesca Orlandi, Ivrea (IT)

(21) Appl. No.: 19/326,122

(22) Filed: Sep. 11, 2025

Related U.S. Application Data
(63) Continuation of application No. 17/622,058, filed on Dec. 22, 2021.

(30) Foreign Application Priority Data
Jul. 2, 2019 (EP) 19184017.2

(51) Int. Cl. A61K 51/04; A61P 35/04

(52) U.S. Cl. CPC A61K 51/0402; A61K 51/0482; A61K 51/0497; A61P 35/04

(57) ABSTRACT

The present disclosure relates to prostate specific membrane antigen (PSMA) ligands. In particular, the disclosure relates to PSMA ligands having a glutamate-urea-lysine (GUL) moiety, a radioisotope and a chelating agent that can comprise a radiometal.

### TECHNICAL FIELD

The present disclosure relates to prostate specific membrane antigen (PSMA) ligands. In particular, the disclosure relates to PSMA ligands having a glutamate-urea-lysine (GUL) moiety, a radioisotope and a chelating agent that can comprise a radiometal.

The disclosure also relates to the use of these compounds in imaging and in the treatment of prostate cancer.

### BACKGROUND ART

Theragnostics is a patient management strategy involving the integration of diagnostics and therapeutics.

In the context of nuclear medicine, theragnostics refers to the use of molecular targeting molecules labeled either with diagnostic radiometal (e.g., positron or gamma emitters), or with therapeutic radiometal (e.g., beta emitters) for diagnosis and therapy of a particular disease. The most developed platform is based upon radiolabeling a targeting molecule, with high affinity for a receptor expressed on a tumor cell, with either gallium Ga-68 for diagnostic use, or lutetium Lu-177 for therapeutic purposes. Therefore, molecular imaging and diagnosis of the disease can be effectively followed by personalized treatment utilizing the same molecular targeting compounds.

Using the same targeting compounds in this manner also enables a more complete approach to patient management, because the diagnostic can then serve several simultaneous functions: diagnosis, restaging, monitoring, selection for therapy using the same molecule labeled with a therapeutic radionuclide, or a different treatment course, as well as follow-up after treatment.

For patients, theragnostics can lead to more effective care, tailoring therapeutic intervention to patients who would benefit the most while reducing or eliminating unnecessary treatment. By predicting patients who might not respond to unnecessary treatments or would otherwise experience side effects, a theragnostic approach is both efficient and patient centric.

For physicians, theragnostics can enhance their ability to diagnose and stage disease, select optimal therapies, and monitor treatment response and disease progression, improving prognostic capability for better health outcomes in a safe and effective manner.

For payors, theragnostic approaches can reduce costs associated with suboptimal diagnostics and treatments and shorten the time needed to diagnose and treat patients with effective individualized treatment plans.

Prostate cancer is one of the most widespread cancers in the US and in Europe. In particular, metastatic prostate cancer (mCRPC) is associated with poor prognosis and diminished quality of life.

Recently, a new development stream for treating prostate cancer is represented by the endo-radiotherapy based on PSMA ligands, as PSMA is considered to be a suitable target for imaging and therapy due to its over-expression in primary cancer lesions and in soft-tissue/bone metastatic disease. Also, PSMA expression seems to be even higher in the most aggressive castration-resistant variants of the disease, which represents a patient population with high unmet medical need. (Marchal et al., Histol Histopathol, 2004 July; 19(3):715-8; Mease et al., Curr Top Med Chem, 2013, 13(8):951-62).

Among many small-molecule ligands targeting PSMA, the urea-based low molecular weight agents have been the most extensively investigated ones. These agents were shown to be suitable for prostate cancer clinical assessment as well as for PRRT therapy. Some of these agents have glutamate-urea-lysine (GUL) as the targeting scaffold.

Several radiolabeled small-molecule inhibitors of PSMA have been designed based on this platform (Kiess A P, Banerjee S R, Mease R C, et al. Prostate-specific membrane antigen as a target for cancer imaging and therapy. Q J Nucl Med Mol Imaging. 2015;59:241-268)

But in certain circumstance, the use of diagnostic radiometal could be limiting (availability of 68Ga, issue due to a decentralized production).

However, 68Ga has a physical half-life of only 68 min. Therefore, 68Ga-PSMA-PET scans are preferably performed in house, and delivery of sufficient tracer activities to remote centers is challenging. Consequently, in large centers with many patients, several productions per day are required, or multiple generators need to be operated simultaneously, multiplying costs. To meet the quantitative demand of those centers, the use of 18F-labeled PSMA tracers may overcome these limitations. PET radiopharmacies with an on-site cyclotron can produce high activities of 18F at moderate costs. The physical half-life (110 min) of 18F-labeled PSMA tracers may also enable centralized production and delivery to distant satellite centers. 18F also has a lower positron energy than 68Ga (0.65 vs. 1.90 MeV), theoretically resulting in an improved spatial resolution.

However, it is always desirable to develop alternative PSMA ligands. It is why the development of alternative multimodal PSMA ligands suitable for their use as diagnostics and/or therapeutics could be advantageous.

### SUMMARY OF THE DISCLOSURE

In a first aspect, the present disclosure relates to a compound of formula (I):

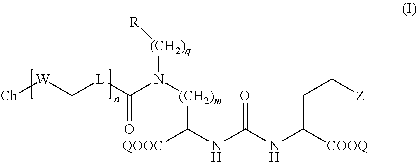

wherein:
Z is tetrazole or COOQ, preferably Z is COOQ:
Q is independently H or a protecting group, preferably Q is H:
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to 10 ring atoms, said aryl and heteroaryl being substituted 1 or more times with X;
X is −Z—Y;
Z is a bond or a C₁-C₆ alkylene, preferably Z is a bond:
Y is a radioisotope:
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR′R′″, —NR″C(O)OR′, —NR″—C(NR″R′″)═NR″″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R′″ each may heteroaryl:
W is selected from the group consisting of —NR²—(C═O), —NR²—(C═S), —(C═O)—NR²—, and —(C═S)—NR²—, preferably, W is —(C═O)—NR²—;
each occurrence of L and W can be the same or different;
R² is H or C₁-C₄ alkyl, preferably R² is H;
n is an integer selected from the group consisting of 1, 2 and 3;
Ch is a chelating agent optionally comprising a metal or a radiometal;
and pharmaceutically acceptable salts thereof.

The compound of formula (I) comprises both a radioisotope and a chelating agent that can comprise a radiometal. The fact that the PSMA ligand may be labeled via different modality allows extending its use. For example, the compound of formula (I) can comprise a radio-halogen used for imaging or therapy purposes in nuclear medicine and a metal.

In a second aspect, the disclosure relates to a pharmaceutical composition comprising a compound of formula (I) and at least one pharmaceutically acceptable carrier.

In a third aspect, the disclosure relates to a compound of formula (I) for use as a drug.

In a fourth aspect, the disclosure relates to a compound of formula (I), for use in treating cancer, especially prostate cancer.

In a fifth aspect, the disclosure relates to a compound of formula (I), for use in imaging.

In a sixth aspect, the disclosure also relates to a method for treating prostate cancer, the method comprising contacting the cancer cells with an effective amount of the compound of formula (I).

In a seventh aspect, the disclosure also relates to a method for imaging, the method comprising contacting the cancer cells with an effective amount of the compound of formula (I) and detecting the signal derived from the decay of the radioisotope present in said compound.

### DETAILED DESCRIPTION

### Definitions

As used herein, the terms “protecting group” in reference to compounds of formula (I) refer to a chemical substituent which can be selectively removed by readily available reagents which do not attack the regenerated functional group or other functional groups in the molecule. Suitable protecting groups are known in the art and continue to be developed. Suitable protecting groups may be found, for example in Wutz et al. (“Greene's Protective Groups in Organic Synthesis, Fourth Edition,” Wiley-Interscience, 2007). Protecting groups for protection of the carboxyl group, as described by Wutz et al. (pages 533-643), are used in certain embodiments. In some embodiments, the protecting group is removable by treatment with acid.

Representative examples of protecting groups include, but are not limited to, benzyl, p-methoxy benzyl (PMB), tertiary butyl (t-Bu), methoxymethyl (MOM), methoxyethoxymethyl (MEM), methylthiomethyl (MTM), tetrahydropyranyl (THP), tetrahydrofuranyl (THF), benzyloxymethyl (BOM), trimethylsilyl (TMS), triethylsilyl (TES), t-butyldimethylsilyl (TBDMS), and triphenylmethyl (trityl, Tr). Persons skilled in the art will recognize appropriate situations in which protecting groups are required and will be able to select an appropriate protecting group for use in a particular circumstance.

As used herein, the terms “alkyl”, by itself or as part of another substituent, refer to a linear or branched alkyl functional group having 1 to 12 carbon atoms. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

Alkylene refers to a divalent alkyl as defined above.

As used herein, the terms “cycloalkyl” refer to a saturated or unsaturated cyclic group having 3 to 6 carbon atoms. Suitable cycloalkyl groups include cyclopropyl, cyclobutyl, cyclopentyl and cyclohexyl.

Cycloalkylene refers to a divalent cycloalkyl as defined above.

As used herein, the term “aryl” refers to a polyunsaturated, aromatic hydrocarbyl group having a single ring or multiple aromatic rings fused together, containing 6 to 10 ring atoms, wherein at least one ring is aromatic. The aromatic ring may optionally include one to two additional rings (cycloalkyl, heterocyclyl or heteroaryl as defined herein) fused thereto. Suitable aryl groups include phenyl, naphtyl and phenyl ring fused to a heterocyclyl, like benzopyranyl, benzodioxolyl, benzodioxanyl and the like.

Arylene refers to a divalent aryl as defined above.

The alkyl, cycloalkyl and aryl monovalent and divalent derivative groups can be substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR′—C(NR″R′″)═NR″″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, alkyl, heteroalkyl, cycloalkyl, heterocycloalkyl, aryl and heteroaryl.

As used herein, the term “halogen” refers to a fluoro (—F), chloro (—Cl), bromo (—Br), or iodo (—I) group.

As used herein, the term “heteroalkyl” refers to a linear or branched alkyl functional group having 1 to 6 carbon atoms and from one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the alkyl group is attached to the remainder of the molecule.

As used herein, the terms “heteroaryl” refer to a polyunsaturated, aromatic ring system having a single ring or multiple aromatic rings fused together or linked covalently, containing 5 to 10 atoms, wherein at least one ring is aromatic and at least one ring atom is a heteroatom selected from N, O and S. The nitrogen and sulfur heteroatoms may optionally be oxidized and the nitrogen heteroatoms may optionally be quaternized. Such rings may be fused to an aryl, cycloalkyl or heterocyclyl ring. Non-limiting examples of such heteroaryl, include: furanyl, thiophenyl, pyrrolyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, indolyl, isoindolyl, benzofuranyl, isobenzofuranyl, benzothiophenyl, isobenzothiophenyl, indazolyl, benzimidazolyl, benzoxazolyl, purinyl, benzothiadiazolyl, quinolinyl, isoquinolinyl, cinnolinyl, quinazolinyl and quinoxalinyl.

As used herein, the terms “heterocycloalkyl” refers to a saturated or unsaturated cyclic group having 5 to 10 ring atoms, wherein at least one ring atom is a heteroatom selected from N, O and S. The nitrogen and sulfur heteroatoms may optionally be oxidized and the nitrogen heteroatoms may optionally be quaternized. Examples of heterocycle include, but are not limited to, tetrahydropyridyl, piperidinyl, morpholinyl, tetrahydrofuranyl, tetrahydrothienyl, piperazinyl, 1-azepanyl, imidazolinyl, 1,4-dioxanyl and the like.

Various embodiments of the disclosure are described herein. It will be recognized that features specified in each embodiment may be combined with other specified features to provide further embodiments.

The present disclosure encompasses the compounds of formula (I), (II), (III) and (IV), their stereoisomers, tautomers, enantiomers, diastereomers, racemates or mixtures thereof, and their hydrates, solvates or pharmaceutically acceptable salts.

The terms “pharmaceutically acceptable salts” refers to salts that retain the biological effectiveness and properties of the compounds of this disclosure and, which typically are not biologically or otherwise undesirable.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

As used herein the term “subject” refers to an animal, preferably a mammal and more preferably a human.

### Compounds of Formula (I)

In a first aspect, the present disclosure relates to compound of formula (I):

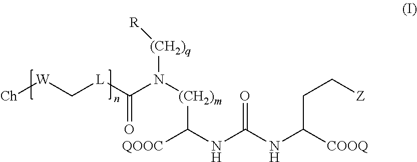

wherein:
Z is tetrazole or COOQ, preferably Z is COOQ;
Q is independently H or a protecting group, preferably Q is H;
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to 10 ring atoms, said aryl and heteroaryl being substituted 1 or more times with X;
X is —Z—Y;
Z is a bond or a C₁-C₆ alkylene, preferably Z is a bond;
Y is a radioisotope;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R″, —NR″C(O)OR′, —NR′—C(NR″R′″)═NR″″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, alkyl, heteroalkyl, cycloalkyl, heterocycloalkyl, aryl and heteroaryl;
W is selected from the group consisting of —NR²—(C═O), —NR²—(C═S), —(C═O)—NR²—, and —(C═S)—NR²—, preferably, W is —(C═O)—NR²—;
each occurrence of L and W can be the same or different;
R² is H or C₁-C₄ alkyl, preferably R² is H;
n is an integer selected from the group consisting of 1, 2 and 3;
Ch is a chelating agent optionally comprising a metal or a radiometal;
and pharmaceutically acceptable salts thereof.

Compounds of formula (I) include the stereoisomers of formulae (Ia), (Ib), (Ic) and (Id):

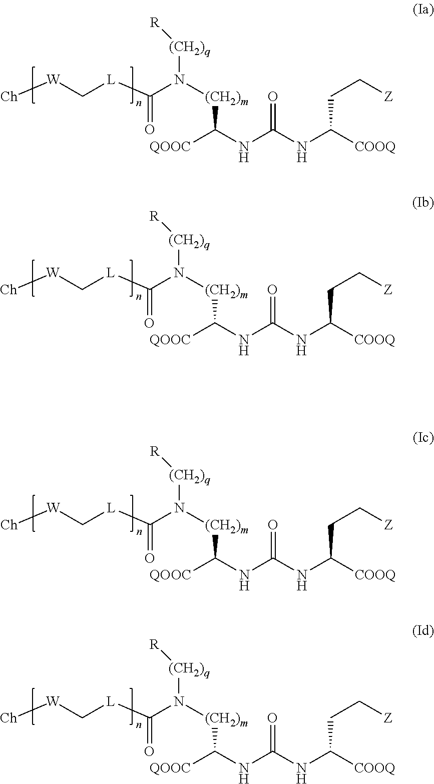

The phrase “wherein each occurrence of L and W can be the same or different” means that when the variable “n” is 2 or 3, one “L” group can be C₁-C₆ alkylene, whereas the other “L” group or groups can be C₃-C₆ cycloalkylene or arylene, or, in other embodiments, each “L” group can be, for example, C₁-C₆ alkylene. Likewise, for example, when “n” is 2 or 3, one “W” group can be —(C═O)—NR²— and the other “W” group or groups can be —(C═S)—NR²—, or, in other embodiments, each “W” can be, for example, —(C═O)—NR²—.

According to an embodiment, L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, —NR′R″, -halogen, —OC(O)R′, —C(O)R′, —CO2R′, —C(O)NR′R″, —OC(O)NR′R″, —NR″C(O)R′, —NR′—C(O)NR″R′″, —NR″C(O)OR′, in a number ranging from zero to (2m′+1), where m′ is the total number of carbon atoms in such groups. R′, R″, R′″ and R″″ each may independently refer to hydrogen, alkyl, heteroalkyl, cycloalkyl, heterocycloalkyl, aryl and heteroaryl.

According to an embodiment, L is a linker selected from the group consisting of C₃-C₆ alkylene optionally substituted with one or more substituents selected from: —OR′, ═O, ═NR′, —NR′R″, -halogen, —OC(O)R′, —C(O)R′, —CO2R′, —C(O)NR′R″, —OC(O)NR′R″, —NR″C(O)R′, —NR′—C(O)NR″R′″, —NR″C(O)OR′, in a number ranging from zero to (2m′+1), where m′ is the total number of carbon atoms in such groups. R′, R″, R′″ and R″″ each may independently refer to hydrogen, alkyl, heteroalkyl, cycloalkyl, heterocycloalkyl, aryl and heteroaryl.

Typically, the radioisotope Y is a radiohalogen and can be selected from the group consisting of ¹⁸F, ³⁴Cl, ⁷⁵Br, ⁷⁶Br, ⁷⁷Br, ¹²³I, ¹²⁴I, ¹²⁵I, ¹³¹I and ²¹¹At, preferably Y is ¹⁸F or ²¹¹At.

According to an embodiment, R is selected from the group consisting of:

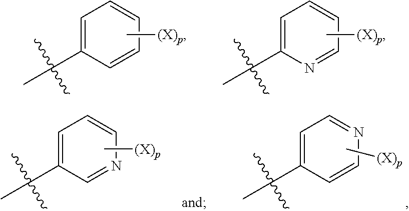

wherein p is an integer selected from the group consisting of 1, 2, 3, 4, and 5, preferably p is 1;
preferably, R is selected from

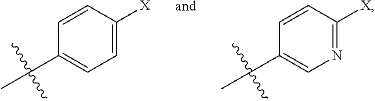

and, more preferably R is

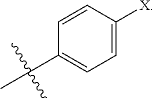

Advantageously, R is

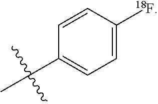

Ch can be selected from the group consisting of:

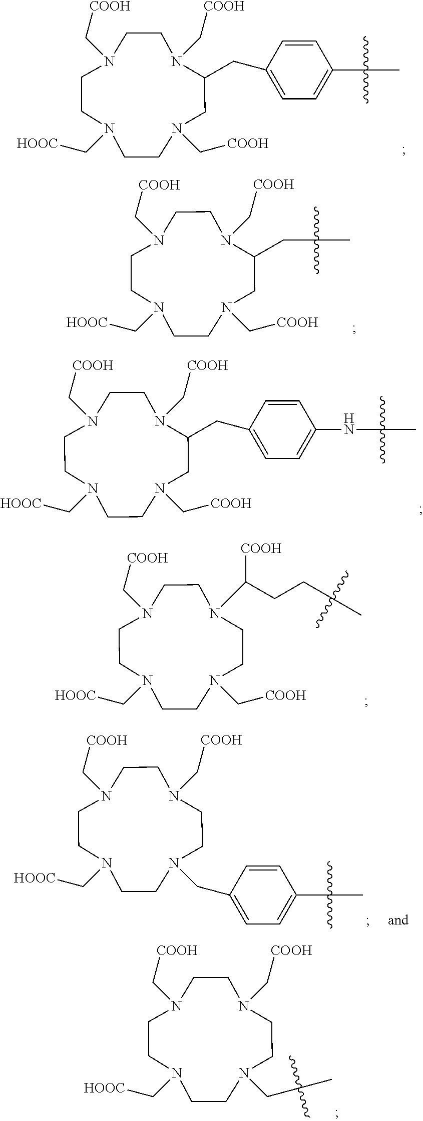

and optionally comprises a metal or a radiometal.

According to a specific embodiment, Ch is

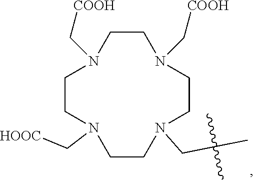 [SMILES: *CN1CCN(CC(=O)O)CCN(CC(=O)O)CCN(CC(=O)O)CC1]

and optionally comprises a metal or a radiometal.

The metal or radiometal is preferably chosen from metal and radiometals that are suitable for use in imaging method or in therapy.

According to an embodiment, Ch comprises a metal selected from Y, Lu, Tc, Zr, In, Sm, Re, Cu, Pb, Ac, Bi, Al, Ga, Re, Ho and Sc. The metal can be a radiometal selected from ⁶⁸Ga, ⁶⁴Cu, ⁸⁶Y, ⁹⁰Y, ⁸⁹Zr, ¹¹¹In, ^{99m}Tc, ¹⁷⁷Lu, ¹⁵³Sm, ¹⁸⁶Re, ¹⁸⁸Re, ⁶⁷Cu, ²¹²Pb, ²²⁵ Ac, ²¹³Bi, ²¹²Bi, ²¹²Pb, ⁶⁷Ga, ²⁰³Pb, ⁴⁷Sc, and ¹⁶⁶Ho.

Advantageously, Ch comprises a radiometal ⁶⁸Ga or ¹⁷⁷Lu.

According to an embodiment, W is —(C═O)—NR²—, and Ch is

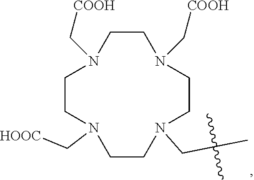 [SMILES: *CN1CCN(CC(=O)O)CCN(CC(=O)O)CCN(CC(=O)O)CC1]

and optionally comprises a metal or a radiometal.

According to an embodiment, m is 4, Z is COOQ, and Q is H.

According to an embodiment, R is

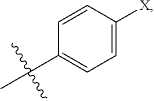

### and Ch is

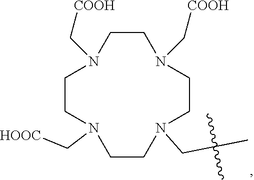 [SMILES: *CN1CCN(CC(=O)O)CCN(CC(=O)O)CCN(CC(=O)O)CC1]

and optionally comprises a metal or a radiometal.

According to a specific embodiment, the compound of formula (I) is the compound of formula (II):

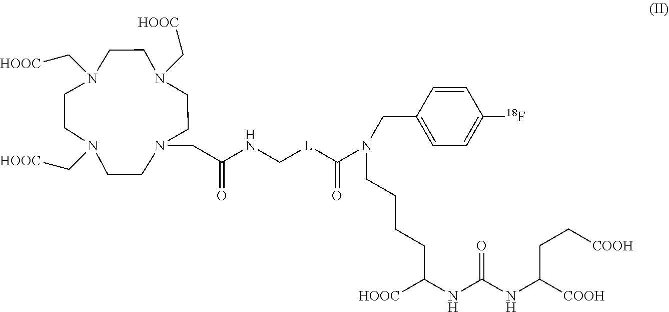

wherein L is a linker selected from the group consisting of C₃-C₆ alkylene optionally substituted with one or more substituents selected from: —OR′, ═O, ═NR′, —NR′R″, -halogen, —OC(O)R′, —C(O)R′, —CO2R′, —C(O)NR′R″, —OC(O)NR′R″, —NR″C(O)R′, —NR′—C(O)NR″R′″, —NR″C(O)OR′, in a number ranging from zero to (2m′+1), where m′ is the total number of carbon atoms in such groups. R′, R″, R′″ and R″″ each may independently refer to hydrogen, alkyl, heteroalkyl, cycloalkyl, heterocycloalkyl, aryl and heteroaryl.

Advantageously, the compound of formula (II) comprises a metal or a radiometal, preferably selected from ⁶⁸Ga, ⁶⁷Ga, ¹⁷⁷Lu, and ¹⁷⁶Lu.

According to a specific embodiment, the compound of formula (II) comprises ⁶⁸Ga or ⁶⁷Ga.

According to another specific embodiment, the compound of formula (II) comprises ¹⁷⁷Lu or ¹⁷⁶Lu.

According to a specific embodiment, the compound of formula (I) is the compound of formula (III):

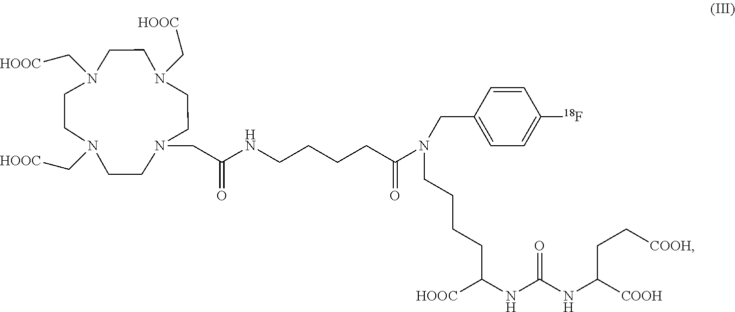 [SMILES: O=C(O)CCC(NC(=O)NC(CCCCN(Cc1ccc([18F])cc1)C(=O)CCCCNC(=O)CN1CCN(CC(=O)O)CCN(CC(=O)O)CCN(CC(=O)O)CC1)C(=O)O)C(=O)O]

which optionally comprises a metal or a radiometal.

Compounds of formula (III) include the stereoisomers of formulae (IIIa), (IIIb), (IIIc) and (IIId):

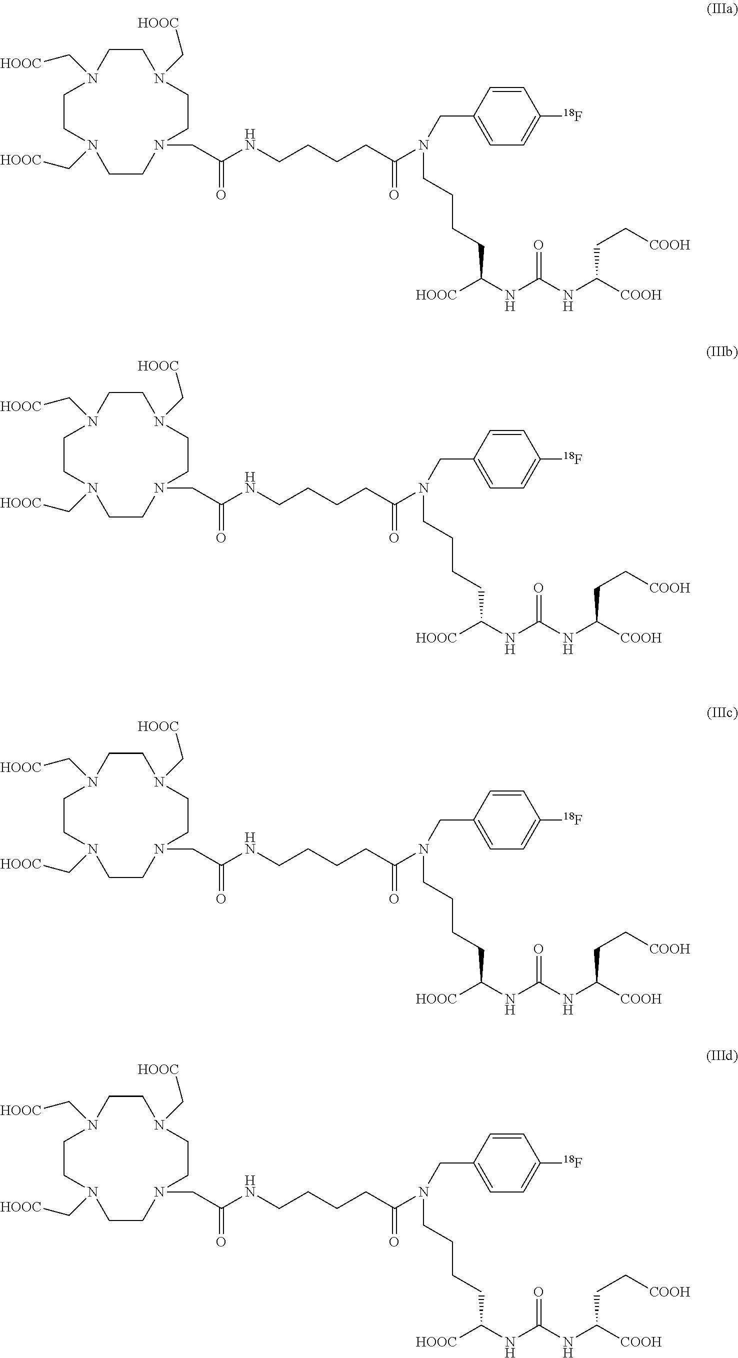 [SMILES: O=C(O)CC[C@@H](NC(=O)N[C@H](CCCCN(Cc1ccc([18F])cc1)C(=O)CCCCNC(=O)CN1CCN(CC(=O)O)CCN(CC(=O)O)CCN(CC(=O)O)CC1)C(=O)O)C(=O)O ; O=C(O)CC[C@H](NC(=O)N[C@@H](CCCCN(Cc1ccc([18F])cc1)C(=O)CCCCNC(=O)CN1CCN(CC(=O)O)CCN(CC(=O)O)CCN(CC(=O)O)CC1)C(=O)O)C(=O)O ; O=C(O)CC[C@H](NC(=O)N[C@H](CCCCN(Cc1ccc([18F])cc1)C(=O)CCCCNC(=O)CN1CCN(CC(=O)O)CCN(CC(=O)O)CCN(CC(=O)O)CC1)C(=O)O)C(=O)O ; O=C(O)CC[C@@H](NC(=O)N[C@@H](CCCCN(Cc1ccc([18F])cc1)C(=O)CCCCNC(=O)CN1CCN(CC(=O)O)CCN(CC(=O)O)CCN(CC(=O)O)CC1)C(=O)O)C(=O)O]

Advantageously, the compound of formula (II) comprises a metal or a radiometal, preferably selected from ⁶⁸Ga, ⁶⁷Ga, ¹⁷⁷Lu, and ¹⁷⁶Lu.

According to a specific embodiment, the compound of formula (III) comprises ⁶⁸Ga or ⁶⁷Ga.

According to another specific embodiment, the compound of formula (III) comprises ¹⁷⁷Lu or ¹⁷⁶Lu.

According to an embodiment, the compound of formula (I) is the compound of formula (IV):

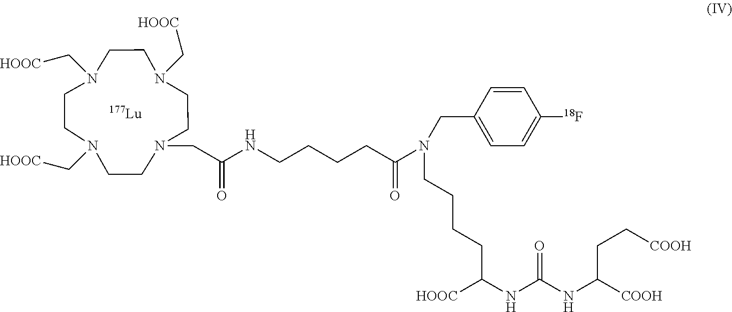

Compounds of formula (IV) include the stereoisomers of formulae (IVa), (IVb), (IVc) and (IVd):

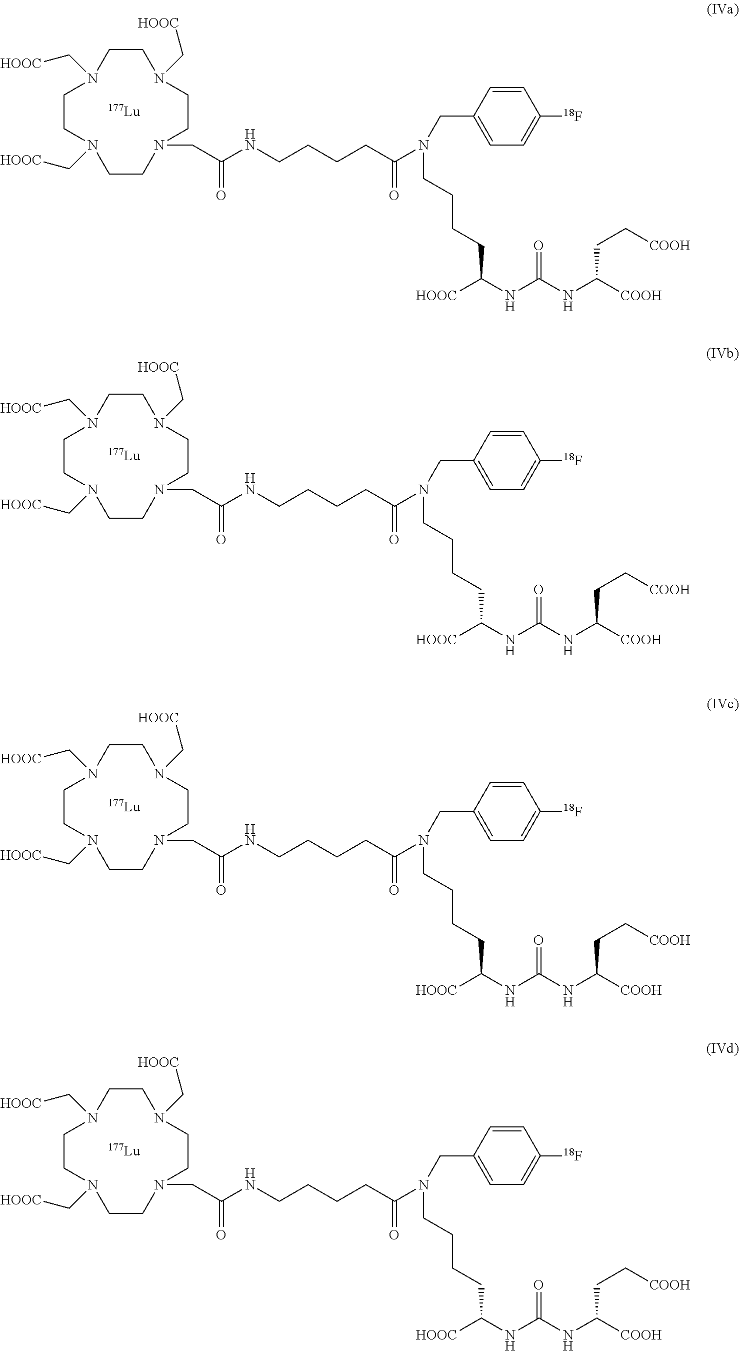

According to another embodiment, the compound of formula (I) is the compound of formula (V):

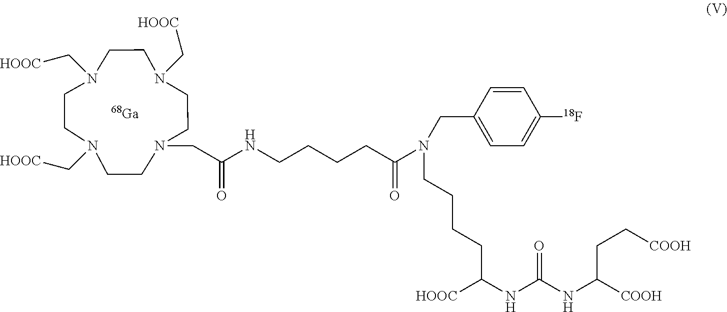

Compounds of formula (V) include the stereoisomers of formulae (Va), (Vb), (Vc) and (Vd):

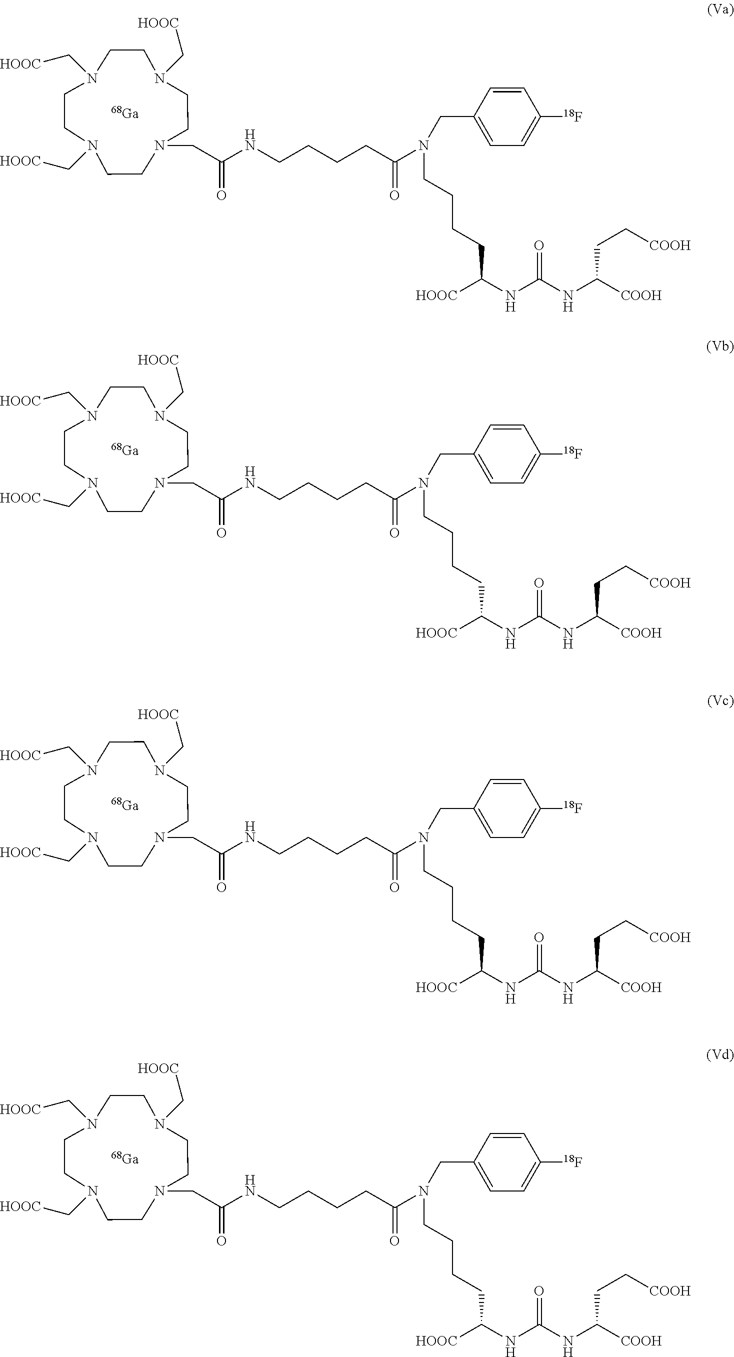

### Pharmaceutical Composition

The disclosure also relates to a pharmaceutical composition comprising a compound of formula (I) to (V) and at least one pharmaceutically acceptable carrier.

The pharmaceutical composition can further comprise a compound comprising i) a PSMA binding ligand, ii) optionally a linker, and iii) a chelating agent optionally comprising a metal or a radiometal.

According to an embodiment, the pharmaceutical composition further comprises a compound of formula (I′), which is a compound of formula (I), wherein Y is a halogen and not a radioisotope. The compound of formula (I′) has the following formula:

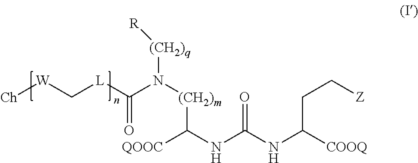

wherein:
Z is tetrazole or COOQ, preferably Z is COOQ;
Q is independently H or a protecting group, preferably Q is H;
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to 10 ring atoms, said aryl and heteroaryl being substituted 1 or more times with X;
X is —Z—Y;
Z is a bond or a C₁-C₆ alkylene, preferably Z is a bond;
Y is a halogen;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR′—C(NR″R′″)═NR″″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may heteroaryl;
W is selected from the group consisting of —NR²—(C═O), —NR²—(C═S), —(C═O)—NR²—, and —(C═S)—NR²—, preferably, W is —(C═O)—NR²—;
each occurrence of L and W can be the same or different;
R² is H or C₁-C₄ alkyl, preferably R² is H;
n is an integer selected from the group consisting of 1, 2 and 3;
Ch is a chelating agent optionally comprising a metal or a radiometal;
and pharmaceutically acceptable salts thereof.

The form of the pharmaceutical compositions, the route of administration, the dosage and the regimen naturally depend upon the condition to be treated, the severity of the illness, the age, weight, and sex of the patient, etc.

The pharmaceutical compositions of the disclosure can be formulated for an intravenous, intramuscular or subcutaneous administration and the like.

The pharmaceutical compositions can take the form of an aqueous solution, for example an injectable formulation comprising at least one compound according this disclosure.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above.

For parenteral administration in an aqueous solution, for example, the solution may be suitably buffered and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In a particular, embodiment the pharmaceutical composition comprises one or more excipient(s) which is selected from stabilizers against radiolytic degradation, buffers, sequestering agents and mixtures thereof.

As used herein, “stabilizer against radiolytic degradation” refers to stabilizing agent which protects organic molecules against radiolytic degradation, e.g. when a gamma ray emitted from the radionuclide is cleaving a bond between the atoms of an organic molecules and radicals are forms, those radicals are then scavenged by the stabilizer which avoids the radicals undergo any other chemical reactions which might lead to undesired, potentially ineffective or even toxic molecules. Therefore, those stabilizers are also referred to as “free radical scavengers” or in short “radical scavengers”. Other alternative terms for those stabilizers are “radiation stability enhancers”, “radiolytic stabilizers”, or simply “quenchers”. As used herein, “sequestering agent” refers to a chelating agent suitable to complex free radionuclide metal ions in the formulation (which are not complexed with the radiolabelled peptide).

Buffers include acetate buffer, citrate buffer and phosphate buffer.

The doses used for the administration can be adapted as a function of various parameters, and in particular as a function of the mode of administration used, of the relevant pathology, or alternatively of the desired duration of treatment. It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient.

### Compound of Formula (I) to (V) for Use as a Drug and Methods Thereof

The disclosure also relates to a compound of formula (I) to (V) for use as a drug. The compounds of formula (I) to (V) exhibit valuable pharmaceutical properties as indicated in the tests provided in the examples and are therefore indicated for therapy.

The disclosure also relates to a compound of formula (I) to (V) for use in treating cancer, in particular by targeted alpha therapy or by beta radiation

Compound of formula (IV) is particularly suitable for use as a drug, preferably for use in a treating cancer.

As used herein, the term “cancer” has its general meaning in the art and includes an abnormal state or condition characterized by rapidly proliferating cell growth. The term is meant to include all types of cancerous growths or oncogenic processes, metastatic tissues or malignantly transformed cells, tissues or organs, irrespective of histopathologic type or stage of invasiveness. The term cancer includes malignancies of the various organ systems, such as affecting skin, lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the oesophages.

Examples of cancer include, but are not limited, to hematological malignancies such as B-cell lymphoid neoplasm, T-cell lymphoid neoplasm, non-hodgkin lymphoma (NHL), B-NHL, T-NHL, chronic lymphocytic leukemia (CLL), small lymphocytic lymphoma (SLL), mantle cell lymphoma (MCL), NK-cell lymphoid neoplasm, and myeloid cell lineage neoplasm. Examples of non-hematological cancers include, but are not limited to, skin cancer, colon cancer, breast cancer, lung cancer, brain cancer, prostate cancer, head and neck cancer, pancreatic cancer, bladder cancer, colorectal cancer, bone cancer, cervical cancer, liver cancer, oral cancer, esophageal cancer, thyroid cancer, kidney cancer, stomach cancer and testicular cancer.

In a specific embodiment, the cancer is a cancer having PSMA expressing tumor or cells.

In a specific embodiment, the disclosure also relates to a compound of formula (I) to (V) for use in a treating prostate cancer.

In a specific embodiment, the prostate cancer is a metastatic prostate cancer.

The disclosure also relates to a compound of formula (I) to (V) for use in treating PSMA-expressing tumors or cells.

The PSMA-expressing tumor or cell can be selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof. In some other embodiments, the PSMA-expressing tumors or cells is a prostate tumor or cell.

Hence, the disclosure also relates to a method for treating cancer, the method comprising contacting cancer cells with a therapeutically efficient amount of the compound of formula (I) to (V).

In a specific embodiment, the cancer to treat is a cancer having PSMA expressing tumor or cells.

The cancer to treat can preferably be a prostate cancer, typically a prostate cancer which includes metastatic prostate cancer.

The disclosure also relates to a method for treating cancer in a subject in need thereof, the method comprising administering to said subject, preferably a human subject, a therapeutically efficient amount of the compound of formula (I) to (V).

As used herein, the term “contacting” means any action which results in at least one compound comprising the therapeutic agent of the presently disclosed subject matter physically contacting at least one cancer cell. Contacting can include exposing the cell(s) or tumor(s) to the compound in an amount sufficient to result in contact of at least one compound with at least one cell or tumor. The method can be practiced in vitro or ex vivo by introducing, and preferably mixing, the compound and cell(s) or tumor(s) in a controlled environment, such as a culture dish or tube. The method can be practiced in vivo, in which case contacting means exposing at least one cell or tumor in a subject to at least one compound of the presently disclosed subject matter, such as administering the compound to a subject via any suitable route. Typically, the compound is administered via the intravenous route.

As used herein, the term “treating” includes reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the terms “therapeutically efficient amount” of a compound refer to an amount of the compound that will elicit the biological or medical response of a subject, for example, ameliorate the symptoms, alleviate conditions, slow or delay disease progression, or prevent a disease.

The disclosure also relates to the use of a compound of formula (I) to (V) or a pharmaceutical composition comprising a compound of formula (I) to (V) and at least one pharmaceutically acceptable carrier, for the manufacture of a medicament for the treatment of cancer, preferably prostate cancer.

The disclosure also relates to the use of a compound of formula (I) to (V) or a pharmaceutical composition comprising a compound of formula (I) to (V) and at least one pharmaceutically acceptable carrier, for the manufacture of a medicament for the treatment of PSMA-expressing tumors or cells.

### Compound of Formula (1) to (V) for Use in Imaging and Methods Thereof

The disclosure also relates to a compound of formula (I) to (V) for use in imaging, preferably in vivo imaging.

The disclosure also relates to a compound of formula (I) to (V) for use in imaging PSMA-expressing tumors or cells, for example prostate tumors or cells in a subject.

Compound of formula (V) is particularly suitable for use in imaging, preferably for use in imaging PSMA-expressing tumors or cells.

In a specific embodiment, the imaging method in which the compound of formula (I) to (V) is used is PET (positron emission tomography) or SPECT (Single photon emission computed tomography).

Hence, the disclosure also relates to a method for imaging, the method comprising contacting cancer cells with an effective amount of the compound of formula (I) to (V).

The disclosure also relates to a method for imaging PSMA-expressing tumors or cells, the method comprising contacting PSMA-expressing tumors or cells with a therapeutically efficient amount of the compound of formula (I) to (V). The method can further comprise a step of detecting the signal derived from the decay of the radioisotope and/or radiometal present in said compound present in said compound.

The disclosure also relates to a method for in vivo imaging PSMA-expressing tumors or cells in a subject, the method comprising administering to said subject, preferably a human, a therapeutically efficient amount of the compound of formula (I) to (V), and detecting the signal derived from the decay of the radioisotope and/or radiometal present in said compound.

In a specific embodiment, the present disclosure provides a method for detecting the presence or absence of PSMA-expressing tumors in a subject, comprising:
(i) administering a compound of formula (I) to (V), e.g. as an intravenous injection in said subject;
(ii) acquiring an image, typically by PET or SPECT imaging; and,
(iii) detecting the presence or absence of PSMA-expressing tumors in said subject.

The disclosure also relates to a compound of formula (I) to (V) for use in diagnostic, typically for use in diagnosing cancer disorders, such as PSMA-expressing cancers. The disclosure also relates to a method for diagnosing and/or detecting cancer cells or PSMA-expressing tumors or cells, for example prostate tumors or cells in a subject, the method comprising administering to said subject, preferably a human, a therapeutically efficient amount of the compound of formula (I) to (V), and detecting the signal derived from the decay of the radioisotope and/or radiometal present in said compound.

### Synthesis of the Compound of Formula (I) to (V)

The compound of formula (III) can be synthesized as disclosed in scheme 1. The p-nitrobenzyl group modified of Glu-Lys urea 2 can be prepared by reductive alkylation of Glu-Lys urea 1 with p-nitrobenzaldehyde in presence of sodium cyanoborohydride in methanol. This type of procedure has been described in the literature (Tykvart et al. (2015) Journal of medicinal chemistry 58, 4357-63). Then, an aliphatic linker, Boc-5-aminovaleric acid can be coupled on the same ε-Lys amine of 2, for example using a base (like N,N-diisopropylethylamine) and a coupling agent (like N,N,N′,N′-Tetramethyl-O-(N-succinimidyl) uronium tetrafluoroborate or 1-[Bis(dimethylamino)methylene]-1H-1,2,3-triazolo [4,5-b]pyridinium 3-oxid hexafluorophosphate), to yield compound 3. Compound 3 can then be deprotected to yield compound 4, for example using an acid like trifluoroacetic acid. Conjugation with commercially available DOTA-NHS ester can be performed to yield compound 5. Finally, compound (III) can be obtained by substitution of the nitro group using ¹⁸F⁻.

The compounds of formula (I), (II) and (III) can be radiolabeled using methods which are commonly used in the field of radiolabeling. In particular, the compound of formula (III) can also be radiolabeled with ¹⁷⁷Lu, to form the compound of formula (IV), using the method described in WO2017/165473. The compound of formula (III) can also be radiolabeled with ⁶⁸Ga, to form the compound of formula (V), using the method described in WO024013.

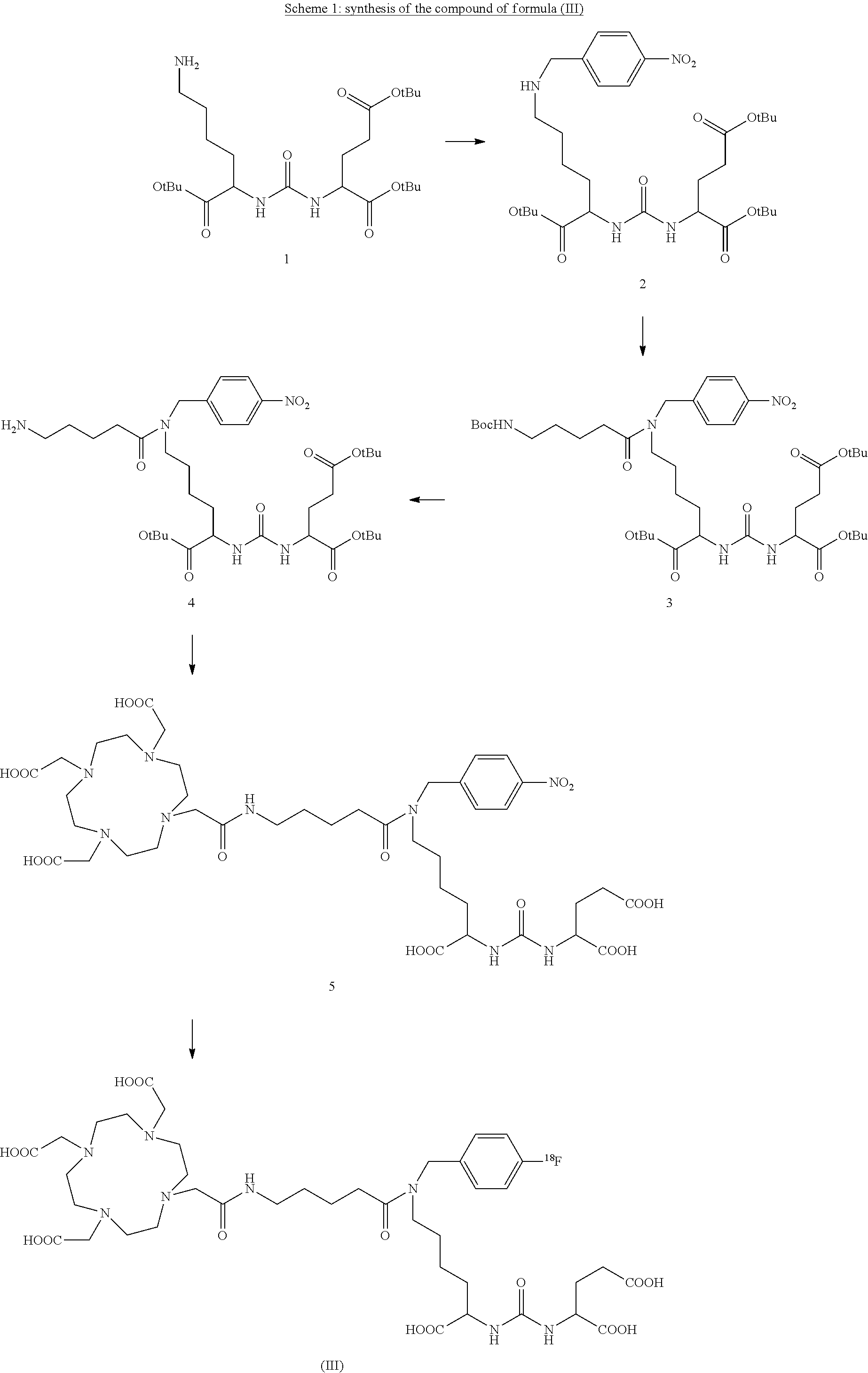

1. A compound of formula (I): 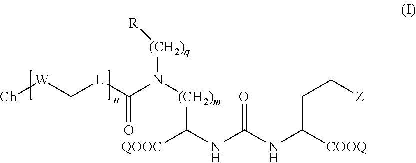
wherein:
Z is tetrazole or COOQ, preferably Z is COOQ;
Q is independently H or a protecting group, preferably Q is H;
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to 10 ring atoms, said aryl and heteroaryl being substituted 1 or more times with X;
X is −Z—Y;
Z is a bond or a C₁-C₆ alkylene, preferably Z is a bond;
Y is a radioisotope;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR′—C(NR″R′″)═NR″″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, alkyl, heteroalkyl, cycloalkyl, heterocycloalkyl, aryl and heteroaryl;
W is selected from the group consisting of —NR²—(C═O), —NR²—(C═S), —(C═O)—NR²—, and —(C═S)—NR²—, preferably, W is —(C═O)—NR²—;
each occurrence of L and W can be the same or different;
R²is H or C₁-C₄ alkyl, preferably R²is H;
n is an integer selected from the group consisting of 1, 2 and 3;
Ch is a chelating agent optionally comprising a metal or a radiometal;
and pharmaceutically acceptable salts thereof.

2. The compound of formula (I) according to claim 1, wherein the compound is a compound of formula (Ia), (Ib), (Ic) or (Id):

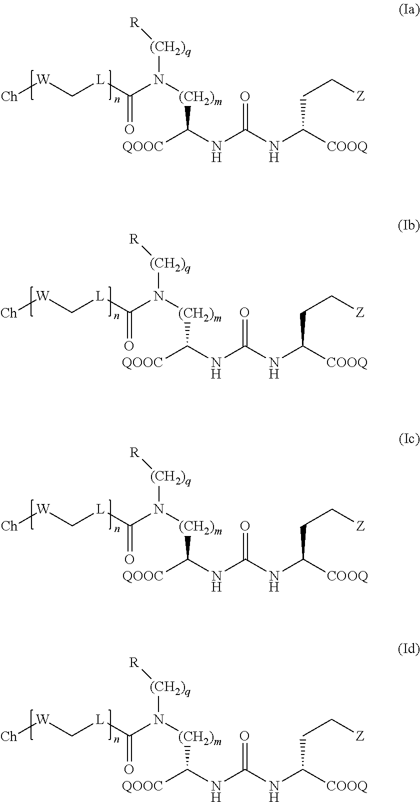

3. The compound of formula (I) according to claim 1 or 2, wherein Y is a radioisotope selected from the group consisting of ¹⁸F, ³⁴Cl, ⁷⁵Br, ⁷⁶Br, ⁷⁷Br, ¹²³I, ¹²⁴I, ¹²⁵I, ¹³¹I and ²¹¹ At, preferably Y is ¹⁸F or ²¹¹ At.

4. The compound of formula (I) according to claims 1 to 3, wherein R is selected from the group consisting of: 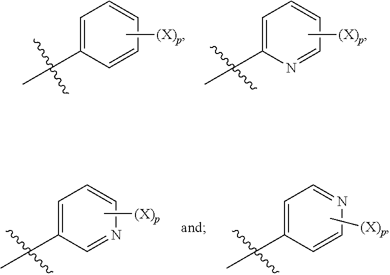
wherein p is an integer selected from the group consisting of 1, 2, 3, 4, and 5, preferably p is 1;
preferably, R is selected from 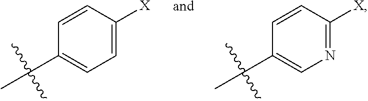
and, more preferably R is

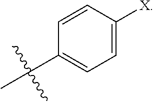

5. The compound of formula (I) according to any one of claims 1 to 4, wherein R is

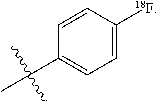

6. The compound of formula (I) according to any one of claims 1 to 5, wherein Ch is selected from the group consisting of: 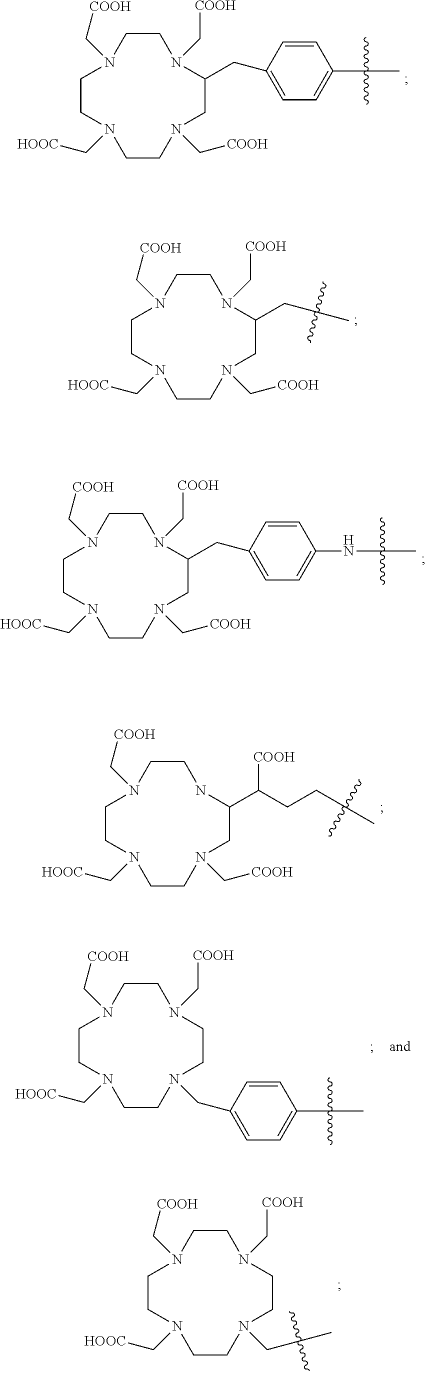 [SMILES: *c1ccc(CC2CN(CC(=O)O)CCN(CC(=O)O)CCN(CC(=O)O)CCN2CC(=O)O)cc1 ; *CC1CN(CC(=O)O)CCN(CC(=O)O)CCN(CC(=O)O)CCN1CC(=O)O ; *Nc1ccc(CC2CN(CC(=O)O)CCN(CC(=O)O)CCN(CC(=O)O)CCN2CC(=O)O)cc1 ; *CCC(C(=O)O)C1CN(CC(=O)O)CCN(CC(=O)O)CCN(CC(=O)O)CCN1 ; *c1ccc(CN2CCN(CC(=O)O)CCN(CC(=O)O)CCN(CC(=O)O)CC2)cc1 ; *CN1CCN(CC(=O)O)CCN(CC(=O)O)CCN(CC(=O)O)CC1]
and optionally comprises a metal or a radiometal.

7. The compound of formula (I) according to any one of claims 1 to 6, wherein L is a linker selected from the group consisting of C₃-C₆ alkylene optionally substituted with one or more substituents selected from: —OR′, ═O, ═NR′, —NR′R″, -halogen, —OC(O)R′, —C(O)R′, —CO2R′, —C(O)NR′R″, —OC(O)NR′R″, —NR″C(O)R′, —NR′—C(O)NR″R′″, —NR″C(O)OR′, in a number ranging from zero to (2m′+1), where m′ is the total number of carbon atoms in such groups. R′, R″, R′″ and R″″ each may independently refer to hydrogen, alkyl, heteroalkyl, cycloalkyl, heterocycloalkyl, aryl and heteroaryl.

8. The compound of formula (I) according to any one of claims 1 to 7, wherein Ch comprises a metal selected from Y, Lu, Tc, Zr, In, Sm, Re, Cu, Pb, Ac, Bi, Al, Ga, Re, Ho and Sc.

9. The compound of formula (I) according to claim 8, wherein the metal is a radiometal selected from ⁶⁸Ga, ⁶⁴Cu, ⁸⁶Y, ⁹⁰Y, ⁸⁹Zr, ¹¹¹In, ⁹⁹mTc, ¹⁷⁷Lu, ¹⁵³Sm, ¹⁸⁶Re, ¹⁸⁸Re, ⁶⁷Cu, ²¹²Pb, ²²⁵Ac, ²¹³Bi, ²¹²Bi, ²¹²Pb, ⁶⁷Ga, ²⁰³Pb, ⁴⁷Sc, and ¹⁶⁶Ho.

10. The compound of formula (I) according to any one of claims 1 to 9, wherein the compound is a compound of formula (II): 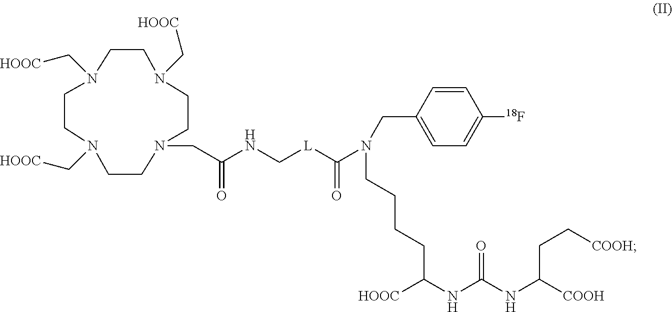
wherein L .is a linker selected from the group consisting of C₃-C₆ alkylene optionally substituted with one or more substituents selected from: —OR′, ═O, ═NR′, —NR′R″, -halogen, —OC(O)R′, —C(O)R′, —CO2R′, —C(O)NR′R″, —OC(O) NR′R″, —NR″C(O)R′, —NR′—C(O)NR″R′″, —NR″C(O)OR′, in a number ranging from zero to (2m′+1), where m′ is the total number of carbon atoms in such groups. R′, R″, R′″ and R″″ each may independently refer to hydrogen, alkyl, heteroalkyl, cycloalkyl, heterocycloalkyl, aryl and heteroaryl.

11. The compound of formula (II) according to claim 10, wherein the compound of formula (II) comprises ⁶⁸Ga or ⁶⁷Ga.

12. The compound of formula (II) according to claim 10, wherein the compound of formula (II) comprises ¹⁷⁷Lu or ¹⁷⁶Lu.

13. A pharmaceutical composition comprising a compound according to any one of claims 1 to 12 and at least one pharmaceutically acceptable carrier.

14. The pharmaceutical composition according to claim 13, wherein said composition further comprises a compound comprising i) a PSMA binding ligand, ii) optionally a linker, and iii) a chelating agent optionally comprising a metal or a radiometal.

15. A compound according to any one of claims 1 to 12 for use as a drug.

16. The compound for use according to claim 15, wherein said compound is for use in treating cancer, in particular by targeted alpha therapy and by beta radiation.

17. The compound for use according to claim 15 or 16, wherein said compound is for use in treating prostate cancer.

18. A compound according to any one of claims 1 to 12 for use in imaging, preferably PET and SPECT.

19. A compound according to any one of claims 1 to 12 for use in diagnostic, typically for use in diagnosing cancer disorders, such as PSMA-expressing cancers.

20. A method for treating cancer, the method comprising contacting cancer cells with a therapeutically efficient amount of a compound according to any one of claims 1 to 12.

21. A method for imaging, the method comprising administering an effective amount of a compound according to any one of claims 1 to 12 to a subject and detecting the signal derived from the decay of the radioisotope and/or radiometal present in said compound.

22. A method for diagnosing and/or detecting cancer cells or PSMA-expressing tumors or cells in a subject, the method comprising administering to said subject, preferably a human, a therapeutically efficient amount of a compound according to any one of claims 1 to 12, and detecting the signal derived from the decay of the radioisotope and/or radiometal present in said compound.
